(19) United States
(12) Patent Application Publication
(10) Pub. No.: US 2015/0343067 A1
(43) Pub. Date: Dec. 3, 2015

(54) Monolithic Dosage Form for the Modified Release of an Active Ingredient Combination

(71) Applicant: HENNIG ARZNEIMITTEL GMBH & CO. KG, Flörsheim am Main (DE)

(72) Inventors: Francas GERNOT, Worms (DE); Karl-Heinz PRZYKLENK, Gross-Gerau (DE)

(73) Assignee: Hennig Arzneimittel Gmbh & Co. Kg,, Florsheim Am Main (DE)

(21) Appl. No.: 14/655,053

(22) PCT Filed: Dec. 23, 2013
(86) PCT No.: PCT/EP2013/077939

§ 371 Date: Jun. 24, 2015

(30) Foreign Application Priority Data
Dec. 27, 2012 (DE) 10 2012 113 098.1
Feb. 1, 2013 (DE) 10 2013 101 049.0

(51) Int. Cl. A61K 47/38; A61K 31/522; A61K 31/138; A61K 31/495

(52) U.S. Cl. CPC A61K 47/38; A61K 31/495; A61K 31/522; A61K 31/138

(57) ABSTRACT

The invention relates to a monolithic peroral dosage form which allows a modified release, preferably an extended and delayed release, of an active ingredient combination of active ingredients with solubility properties that strongly deviate from one another. The dosage form comprises at least one emulsifier with a polyalkylene oxide structural motif and at least one controlled-release agent. Using the dosage forms according to the invention, even active ingredients which have a limited storage stability and other unfavorable active ingredient properties that additionally hinder processing can be featured in an optimal manner. The invention also relates to a method for producing the dosage form and to the use thereof.

This invention relates to monolithic dosage forms for the release of a combination of active ingredients which are suitable for the modified release of active ingredients with solubility properties that strongly deviate from one other. For this, the dosage form comprises at least one first active ingredient with low solubility and one second active ingredient with higher solubility. In particular such active ingredients with low solubility the solubility of which decreases with increasing pH values profit from the dosage forms according to the present invention.

The dosage forms according to the present invention allow an optimal modified release of the combination of active ingredients so that an optimal therapeutic effect and duration of action are guaranteed, even in the case of an intake two times or one time a day of a dosage form according to the present invention, wherein at the same time the risk of dose dumping, in particular of the second active ingredient, is considerably decreased. The dosage forms allow an optimal release of the active ingredients and at the same time the prevention of too high plasma levels and also of too low plasma levels independent on the state, fasting or not fasting (called “saturated state” according to the present invention), in which the intake of the dosage form takes place. In addition, the dosage forms can be produced easily and cheaply, and they are characterized by very good storage stability, wherein the requirements of current pharmacopoeias with respect to the quality of the dosage forms are at least fulfilled as well as preferably exceeded.

Basically, an active ingredient has to be dissolved at first in the physiological milieu of the gastrointestinal tract, so that it can be resorbed and enter the bloodstream as well as induce the desired therapeutic effect. When it is not dissolved, then it is not released from its dosage form, and thus this cannot result in an induction of the therapeutic effect.

A lot of pharmaceutically active ingredients are characterized by low solubility in the physiological milieu of the gastrointestinal tract. For the classification of the active ingredients according to their solubility and permeability normally the Biopharmaceutics Classification System (BCS) is used, according to which there are four classes of active ingredients. According to this classification the solubility is high, when the ratio of the highest single oral dose of the active ingredient (in mg) to its solubility (in mg/ml) is lower than 250 ml over the physiological pH-range of pH 1 to 7.5 at 37° C. Thus, the active ingredient has to dissolve completely in its highest single therapeutic dose in 250 ml of buffer liquid. A high permeation capability is defined as a resorption of the active ingredient of more than 90% in the small intestine. With respect to the method for the determination of the solubility and permeation properties reference is made to the guidelines of the American Food and Drug Administration (FDA) (WHO 2005: Proposal to waive in vivo bioequivalence requirements for the WHO model list of essential medicines immediate release, solid oral dosage forms; Working document QAS/04.109/Rev. 1). Active ingredients which do not fulfil the criteria of high solubility are classified into the BCS classes II and IV. Active ingredients of the BCS class II show high permeability in contrast to such ones of the BCS class IV which besides the low solubility are also characterized by low permeability.

Thus, the resorption of active ingredients of the BCS class II is mainly controlled by the solubility and/or the dissolution rate of the active ingredient over the physiological pH range. In particular in the case of such active ingredients it is important to increase the dissolution of the active ingredient under physiological conditions.

Particular difficulties in addition result from active ingredients having low solubility which in addition show a pH dependent solubility. A pH dependent solubility of the active ingredient is characterized by a dependency of the solubility properties of the active ingredient on the pH value, thus, when the solubility of the active ingredient changes with the pH value. In particular basic functional groups such as amino groups, guanidine groups and nitrogen-containing heterocycles in the molecule of the active ingredient can result in pH dependent solubility properties and thus hinder the formulation of the active ingredient.

In particular the formulation of active ingredients which are weak organic acids or weak organic bases causes difficulties. Weak organic acids comprise acid functions. Therefore, in the intestine they are deprotonated and thus charged. Therefore, they show good solubility in the basic milieu of the intestine. Weak organic bases are not protonated in this basic milieu and thus they are not charged. Weak organic bases contain basic functional groups with a pK_{A} value of at most 9, in particular a pK_{A} value of at most 8. This means, that the corresponding acid of the basic functional group each has the mentioned pK_{A} value. For the weak organic bases it is true that they typically show good solubility in acidic milieu. The basic group in the respective molecule is protonated in an acidic milieu so that the weak organic base is positively charged. Such an acidic milieu can typically be found in the fasting human stomach.

Thus, weak organic bases show poor solubility in the pH range of the intestine. But the intestine, in particular the small intestine, is the place at which typically the resorption of active ingredients takes place and should take place, in particular due to the large resorption area which is provided by the intestine.

The above outlined circumstances of good solubility in the fasting stomach on the one hand and poor solubility in the intestine on the other hand, in addition, hinder the formulation of such active ingredients, in particular in the case, when modified release is desired. It has to be considered that the pH value in the human stomach is not always very acidic. After the ingestion of a meal the pH value may definitely reach neutral values. Only with time the pH value becomes lower again. This means for an active ingredient with weakly basic properties, when its intake is conducted after a meal, that it at first shows poor solubility in the stomach, which improves with time, since the pH value in the stomach becomes more acidic again.

It is difficult to achieve a modified release, in particular an extended release of active ingredients with low solubility. An approach for solving the outlined problem is the formulation of dosage forms with extended release, wherein the dosage form is provided with a coating which releases the active ingredient only slowly. There are also approaches of dispersing such active ingredients in a matrix which is characterized by slow erosion.

In the case of weakly basic active ingredients there is the additional problem that the active ingredient shows only poor solubility in the alkaline milieu of the intestine—as already described. But for an extended, preferably linear release it is required that the active ingredient is dissolved in a continuous manner. In practice this results in the fact that dosage forms which should release a weakly basic active ingredient with poor solubility in the intestine in an extended manner at first comply with these requirements (in the stomach), but when the dosage form reaches the intestine, then such a bad release is achieved that the application is not expedient.

A further problem arises, when two active ingredients with dissolution behaviors that strongly deviate from one another should be released from one dosage form in the same manner, in particular in a modified manner. In the case of better soluble active ingredients, in particular such ones which are permanently charged, there is the problem that the release of these active ingredients has to be delayed. In particular in the saturated state there is the risk that the better soluble active ingredient is released in the stomach too quickly in an amount which is too high (dose dumping), which may result in toxic plasma levels as well as a possible irritation of the gastric mucosa. This is particularly dangerous, when the better soluble active ingredient in addition is characterized by a long half-life of elimination, i.e. a half-life of elimination of longer than 2 hours. Thus, with respect to the active ingredient with low solubility a stimulation of the dissolution and the release, at least in the intestine, has to be achieved and with respect to the better soluble active ingredient controlled release (retardation) has to be achieved. In this case it has to be guaranteed that a sufficient amount of the active ingredient with lower solubility is preferably dissolved in the stomach. But in such a case the stimulation of the dissolution of this active ingredient must not be too excessive, because also then, in particular in the case of a longer residence time in the stomach, this may result in an accumulation of high amounts of active ingredient, which may result in a too steep increase of the plasma level and an irritation of the gastric mucosa.

A further challenge is the provision of a combination of active ingredients with solubility properties that strongly deviate from one another so that an administration several times a day is optimally mimicked and thus the dose intervals can be reduced, ideally to an intake of the dosage form two times or one time a day. Here, especially in the case of long-term therapies a slow and permanent increase of the plasma levels of the active ingredients from the dosage form may be desired, also for avoiding an accumulation with plasma levels of the previous dose in the blood and for avoiding peaks of plasma levels or strongly fluctuating plasma levels.

Additional difficulties arise, when the active ingredients are not characterized by unlimited storage stability and/or besides the unfavorable solubility properties have other active ingredient properties which in addition hinder the processing. A compound is not characterized by unlimited storage stability, when its storage requires special precautionary measures, such as for example the requirement of a storage under light-protection and/or the requirement of a storage of the active ingredient in a tightly closed container. Other active ingredient properties which in addition hinder the processing and thus result in further particular requirements for dosage formulations are for example a bad taste or unpleasant odor of an active ingredient.

Hitherto existing dosage forms in which several active ingredients with solubility properties that strongly deviate from one another are processed continuously require laborious production processes comprising numerous process steps, and therefore the dosage forms are not cheap and cannot be produced quickly. Mostly the active ingredients with the solubility properties that deviate from one another are separately processed and are provided in separated dosage forms or in multi-particulate dosage forms in different subunits, because the support of the release of the active ingredients requires different compositions each. Only in few cases the active ingredients with solubility properties that deviate from one another are provided in one single subunit, however in such multi-particulate dosage forms several subunits with different release behavior are required for designing the release of the active ingredients in such a manner that dose intervals can be extended. The last one requires a laborious multi-step production. In addition, such dosage forms are normally relatively large and therefore this may result in swallowing problems for the patient. This may have a negative influence onto the compliance of the patient.

Thus, there is a need for dosage forms which guarantee a therapeutically optimal modification of the release of at least two active ingredients with solubility properties that strongly deviate from one another in a similar manner and thus considerably reduce the risk of dose dumping of the active ingredients. Here it should be possible, to optimally release even weakly basic active ingredients. In this context “to release” means that the active ingredient is provided, hence made available at the site of resorption, thus in the small intestine. It should be possible that the dosage forms can be produced in a simple and cheap manner, and they should be characterized by optimal storage stability, even in the case of active ingredients with limited storage stability, and they should have a suitable size. In addition, it should be possible that even active ingredients which have other unfavorable active ingredient properties with respect to processing can be provided in an optimal manner. In particular, the dosage forms should allow a reduction of the number of daily doses of active ingredients and thus result in an improved compliance.

The above-described objects are solved by the subject-matter of the patent claims. According to the present invention are dosage forms comprising at least one first active ingredient and one second active ingredient, wherein the first active ingredient preferably comprises at least one basic functional group.

The first active ingredient at a pH value of 7 and 22° C. has a solubility of less than 0.01 mg/ml. The second active ingredient at a pH value of 7 and 22° C. has a solubility which exceeds the solubility of the first active ingredient at a pH value of 7 and 22° C. in an extent of at least one decimal power.

In addition, the dosage forms according to the present invention comprise at least one emulsifier, wherein the emulsifier has a structural motif of the following formula

R¹—O—[CH₂—CH₂—O]ₙ—R²

wherein R¹ and R² independently from each other are hydrogen, alkyl, glyceride or polyalkylene oxide and n is an integer of at least 4. Furthermore, in the dosage forms according to the present invention at least one controlled-release agent (retarding agent) is contained in such a proportion that the mass ratio of the second active ingredient to the controlled-release agent is lower than 1:1. The sum of the proportions of the controlled-release agent and the emulsifier of the dosage form according to the present invention is at least 40% by weight.

According to the present invention, the dosage forms are peroral solid dosage forms. This means that they are intended for oral intake. Peroral solid dosage forms can easily be administered by the patient her—respectively himself, and such dosage forms enjoy a very well acceptance by the patients. The dosage forms according to the present invention may for example be a capsule or a tablet, preferably a tablet.

The dosage forms according to the present invention are monolithic dosage forms. According to the present invention, these dosage forms are dosage forms which during passage through the stomach and the intestine become smaller and smaller by erosion and/or degradation. Monolithic dosage forms are different from multi-particulate dosage forms which after intake or in the stomach decompose into several discrete subunits with a size of normally lower than 2 mm, such as mini-tablets or pellets, with specific properties and release profiles each, which can pass the pylorus sphincter also in the closed state. In contrast thereto, monolithic dosage forms reside in the stomach so long, till their size has become so small by erosion and/or dissolution that the dosage form can pass the pylorus sphincter and/or the stomach is emptied by means of so-called housekeeper contractions. The design as a monolithic dosage form allows a quick and simple as well as cheap production, in contrast to methods for the production of multi-particulate dosage forms. In this context it has to be considered that of course laborious production methods always also strain the active ingredients and can promote their decomposition or generally the formation of impurities. Thus, monolithic dosage forms are not multi-particulate dosage forms. In particular, the dosage form according to the present invention is not prepared by compressing subunits of equal size with specific properties and release profiles each, in particular pellets. The monolithic dosage form according to the present invention after intake in the stomach does not decompose into subunits of equal size, in particular pellets.

In particularly preferable embodiments the dosage forms which are described here are forms with long residence time in the stomach. This means that the dosage forms do not strongly dissolve and/or erode in the stomach so that they can quickly leave the stomach through the pylorus sphincter. Preferably, a form with long residence time in the stomach is such a form which resides in the stomach of the patient for at least 2 hours, more preferably at least 3 hours, before it enters the intestine. According to the present invention this is achieved by the special design of the dosage forms which results in very slow dissolution. So over a longer period of time the size of them is such that they cannot leave the stomach.

Since preferably the dosage forms only dissolve very slowly, they preferably stay in the stomach, until it is emptied nearly completely. Then a large proportion of the chyme has entered the small intestine via the pylorus sphincter. In such a moment the stomach is emptied by means of so-called housekeeper contractions. So also bodies which normally do not pass the pylorus sphincter can reach the small intestine. Then, the dosage forms further release the active ingredients in the small intestine. So a nearly linear release of the first and the second active ingredients becomes possible.

The dosage forms according to the present invention release the first active ingredient and the second active ingredient in a modified manner. According to the present invention, “modified release” means in this case a delayed, pulsatile, extended release of the active ingredients or mixtures of these release kinds. A delayed release means a delay in time of the release of the active ingredient after the intake in comparison to immediately releasing dosage forms, thus a later start of the release. Preferably, this is the case, when after 60 minutes an amount of lower than 30% of the active ingredient is released into the respective test medium, preferably lower than 25%, more preferably lower than 23% and particularly preferably lower than 20%. It is particularly preferable, when after 120 minutes at most 40%, preferably at most 35% and particularly preferably at most 30% of the active ingredient are released from the dosage form. The measurement can be conducted according to common methods of measuring a release, in particular according to the methods of the European Pharmacopeia or the American Pharmacopeia (USP), preferably with the Bio-Dis apparatus (200 ml test medium, 25 dpm, 37° C.).

A person skilled in the art is capable of selecting the test medium in dependence on the active ingredient and the special release kinetics. For example, test media of well-established pharmacopoeias, in particular of the European Pharmacopoeia or the American Pharmacopoeia, can be used. It was found that test media selected from 0.1 N (0.1 mol/l) hydrochloric acid solution (Ph. Eur. 7), FeSSIF buffer according to Dressman et al., FaSSIF buffer according to Dressman et la., buffer solution pH 3.0 R (Ph. Eur. 7), FaSSGF buffer according to Dressman et al. and FeSSGF buffer according to Dressman et al. are particularly suitable for the determination of the release of the dosage form according to the present invention. Preferably, at least two of the mentioned test media are combined for the determination of the release of the dosage form according to the present invention. The combination of test media means that they are used one after the other. Thus, one test medium after a certain time, for example after 60 minutes, is replaced by another test medium. This allows an optimal mimicking of physiological processes after the intake of a medicine. So the results are nearly comparable with the in vivo release of the active ingredient.

A pulsatile release comprises the release of the active ingredient in more than one phase, wherein one phase is one release pulse.

An extended release, according to the present invention, is a release of the active ingredient over at least 5 hours, more preferably at least 6 hours, still more preferably at least 8 hours and ideally even at least 9 hours. Thus, also after at least 5, more preferably at least 6, still more preferably at least 8 and ideally at least 9 hours active ingredient is released from the dosage form into the test medium. So, on the one hand, a therapeutic plasma level for a sufficient period of time during the night can be guaranteed so that the patient is not forced to an intake of a dose during the night. Also the dose intervals during the day can considerably be extended which has a positive influence onto the compliance of the patient.

Preferably, the dosage form according to the present invention releases the active ingredients in an extended manner in combination with a delayed release of the active ingredients. Thus, the release of the first active ingredient and the release of the second active ingredient are started in a delayed manner and at the same time they are extended. Thus, over a longer period of time sufficiently high plasma levels are maintained. Therefore, an administration of the first and/or the second active ingredients several times a day can be reduced by the intake of the dosage form according to the present invention to an intake two times a day, preferably to an intake one time a day.

Preferably, the modified release of the first and the second active ingredients occurs independently from each other, i.e. the release of the first and the second active ingredients is substantially controlled by different mechanisms of release within the dosage form. The emulsifier is involved in the release of the first active ingredient, whereas in the release of the second active ingredient both, the controlled-release agent and the emulsifier are involved. This results in the advantage that the risk of dose dumping of the second active ingredient, in particular in the case of a longer residence time in the stomach, can be reduced more considerably and that the dissolution of the first active ingredient in the stomach can be optimally supported.

The dosage forms according to the present invention are characterized by an extended residence time in the stomach so that they are present in the stomach for a sufficient period of time so that a significant proportion of the first active ingredient can be dissolved, but at the same time dose dumping of the first and the second active ingredients is prevented.

The dosage forms according to the present invention are preferably designed such that their mass does not exceed a value of 1400 mg, more preferably 1200 mg and particularly preferably 1000 mg as well as especially preferably 900 mg. Due to the formulation their mass is preferably at least 400 mg, more preferably at least 450 mg and still more preferably at least 500 mg.

The dosage form of this invention may be designed in the form of a tablet or a capsule. Preferable forms are tablets. Preferably, the dosage form has at least at one site a diameter of more than 5 mm, in particular more than 7 mm and particularly preferably at least 8 mm. So it is guaranteed that the dosage form cannot immediately leave the stomach through the pylorus sphincter, but maintains in the stomach for a certain period of time, preferably at least 2 hours, more preferably at least 3 hours.

After the ingestion of food the stomach is not emptied immediately, but it step by step releases the chyme into the small intestine. This physiological fact can be used with the dosage form according to the present invention. The first active ingredient is preferably a weak base and thus, at a low pH value, thus in the acidic milieu, it shows a better solubility. Typically, in the stomach the pH value is low. But after a meal the pH value increases to higher values, e.g. about pH 5. Only step by step the pH value decreases to lower values again. This means that the first active ingredient after the ingestion of food would at first be dissolved poorly, because the pH value is too high. But the dosage form according to the present invention now allows the dissolution of a part of the first active ingredient by the measures which are described herein.

Through the motion of the stomach the released first active ingredient is mixed with the chyme and is then gradually released into the small intestine. Then the active ingredient is resorbed in the proximal small intestine, thus a short time after leaving the stomach. So the stomach serves as an additional controlled-release agent for the first active ingredient. At the same time also the second active ingredient with better solubility is dissolved.

When the pH value in the stomach has reached a lower value, e.g. about pH 3, then the first active ingredient which is preferably a weak base is dissolved better. Then there is the risk that too much of the first active ingredient is dissolved. Therefore, the release of the first active ingredient in the dosage form according to the present invention is preferably delayed and decelerated. The emulsifier is involved in the delay and deceleration of the release of the first active ingredient, wherein preferably the release of the first active ingredient is controlled by both, the motion of the stomach and also the emulsifier.

It is particularly preferable, when also the release of the second active ingredient is a delayed and extended release. The emulsifier and the controlled-release agent are involved in the modification of the release of the second active ingredient, wherein preferably the release of the second active ingredient is controlled by the emulsifier and the controlled-release agent.

The first active ingredient shows in aqueous solution at a pH value of 7 and at 22° C. a solubility of lower than 0.01 mg/ml. Thus, the first active ingredient has a low solubility, which means that it is “virtually insoluble” in accordance with the solubility classification of the European Pharmacopoeia (Ph. Eur. 7, 2011). Preferably, at a pH value of 7 and at 22° C. the solubility in aqueous solution is at most 0.005 mg/ml, more preferably at most 0.001 mg/ml and still more preferably even lower than 0.001 mg/ml.

Solubility means the concentration of the dissolved substance in a saturated solution at a certain temperature. According to the present invention, the solubility of the active ingredient relates to the solubility in aqueous solution at 22° C. under normal pressure. According to the present invention, this information with respect to the temperature as well as the whole information with respect to the temperature which follows comprise the concretely mentioned value including a span of +/−3° C. around the mentioned temperature value. Information with respect to the solubility which follows relates to the solubility under normal pressure, unless otherwise stated. The determination is conducted in standard and/or buffer solutions of the European Pharmacopoeia (Ph. Eur. 7) with the corresponding pH value.

The aqueous solution for achieving a pH value of 7 comprises a phosphate buffer and water. The composition can be found in the European Pharmacopoeia (Ph. Eur. 7; phosphate buffer solution pH 7.0 R). The measurement of the pH value is conducted according to methods which are known by a person skilled in the art, for example by means of a commercially available pH meter.

For the determination of the solubility methods which are known by a person skilled in the art are used, such as for example the sediment method, the temperature method or the distribution method. The distribution method in which the active ingredient is dissolved in a second solvent which is immiscible with the respective aqueous solution for which the solubility of the first active ingredient should be determined is particularly suitable. Both liquids are intensively shaken until the establishment of equilibrium is achieved and then they are separated.

The first active ingredient is preferably selected from the BCS class II or the BCS class IV, wherein it is particularly preferable, when the first active ingredient is an active ingredient of the BCS class II. In particular such active ingredients especially profit from the dosage forms according to the present invention, because the dissolution of the first active ingredient is particularly supported.

Preferably, the first active ingredient comprises at least one basic functional group. Up to now, especially such functional groups which normally are associated with pH dependent solubility result in considerable difficulties during the formulation of dosage forms.

In particular, a group is basic, when in the uncharged state of the active ingredient it is capable of acquiring protons. In the sense of the invention only groups with a pK_{A} value of at most 9, more preferably a pK_{A} value of at most 8.5 and more preferably a pK_{A} value of at most 8 are called basic functional groups. This means that the respective corresponding acid of the basic functional group is characterized by such a pK_{A} value.

Preferably, the basic functional group is selected from aliphatic nitrogen-containing groups or a nitrogen-containing heterocycle. In the sense of this invention the term “nitrogen-containing heterocycle” means nitrogen-containing groups which are a part of aromatic hydrocarbons. Besides the nitrogen-containing groups the aromatic hydrocarbons may comprise further heteroatoms as ring members. But preferable “nitrogen-containing heterocycles” are such ones which besides nitrogen only contain carbon atoms as ring members. Aliphatic nitrogen-containing groups are preferably selected from amino groups and guanidine groups. The term “amino group” comprises primary, secondary and tertiary amino groups. Also nitrogen atoms in saturated or partially unsaturated cyclic hydrocarbons which besides the nitrogen atom may comprise further hetero atoms including further nitrogen atoms as ring members are an amino group.

Especially preferably, the basic functional group is selected from amino group, guanidine group and nitrogen-containing heterocycle. It is still more preferable, when the basic functional group is an amino group. Such active ingredients particularly profit from the dosage forms according to the present invention due to their marked pH dependent solubility.

It is preferable that at least one basic functional group of the first active ingredient has a pK_{A} value of at least 4.5, more preferably of at least 5.8 and more preferably of at least 6.5. This means that the corresponding acid of the basic functional group is characterized by such a pK_{A} value. Such active ingredients show particularly marked pH dependent solubility properties which till today have particularly hindered a formulation.

Active ingredients which are polybasic, thus comprise at least two basic functional groups profit from the dosage form according to the present invention in an particular manner. Therefore, the first active ingredient particularly preferably contains at least two basic functional groups, still more preferably exactly two basic functional groups. It is still more preferable, when the first active ingredient comprises two amino groups.

Thus preferably, the first active ingredient is a weakly basic active ingredient, thus an active ingredient which comprises at least one basic functional group with a pK_{A} value of at most 9, more preferably of at most 8.5 and more preferably of at most 8. As already mentioned, the information with respect to the pK_{A} value relates to the corresponding acid of the basic functional group.

Therefore, the first active ingredient, when it comes in contact with the chyme, is normally protonated and thus water-soluble in this acidic milieu. At higher pH values in the intestine of 6 and more, however, the active ingredient shows only poor solubility. This in particular applies to active ingredients without any acidic groups.

Thus more preferably, the first active ingredient does not contain any acidic groups. Acidic groups are such ones which in the uncharged state of the active ingredient can donate protons. In particular the first active ingredient does not comprise any acidic groups with a pK_{A} value of lower than 5. Acidic groups in particular comprise carboxyl groups, sulfur-containing acid groups such as sulfonic acid groups and sulfuric acid esters, phosphor-containing acid groups, hydroxyl groups including phenolic hydroxyl groups and amide groups. In particular, no carboxyl groups and/or hydroxyl groups are contained in the first active ingredient. Thus it is particularly preferable, when the solubility of the first active ingredient is substantially affected by the basic functional group, preferably selected from amino group, guanidine group and nitrogen-containing heterocycle, especially preferable by the at least one amino group. Anyhow, such active ingredients particularly profit from the design of the dosage form according to the present invention.

Preferably, the first active ingredient at pH values of <7 and at 22° C. shows higher solubilities than at a pH value of 7 and at 22° C. Preferably, the first active ingredient at a pH value of 1 and 22° C. shows in aqueous solution a solubility of higher than 0.1 mg/ml, more preferably at least 0.5 mg/ml, more preferably at least 1 mg/ml, still more preferably at least 1.3 mg/ml and particularly preferably at least 1.5 mg/ml. Thus, the active ingredient at a pH value of 1 is preferably characterized by at least “very poor solubility”, more preferably by at least “poor solubility” according to the solubility classification of the European Pharmacopoeia (Ph. Eur. 7). Preferably, at pH values of higher than 7 the solubility of the first active ingredient is at most 0.01 mg/ml, more preferably at most 0.001 mg/ml. For example, the solubility of the active ingredient at a pH value of 9 in aqueous solution and at 22° C. is preferably at most 0.01 mg/ml and more preferably at most 0.001 mg/ml.

As the aqueous solution for achieving a pH value of 1 the standard hydrochloric acid solution of the European Pharmacopoeia (Ph. Eur. 7) consisting of hydrochloric acid and water, i.e. a 0.1 N (0.1 mol/l) hydrochloric acid solution, is used. For the determination of the solubility of the active ingredient at a pH value of 9 the phosphate buffer solution pH 9.0 R is suitable.

Preferably, the first active ingredient used according to the present invention has an n-octanol/water distribution coefficient (log P_{OW}) at 20° C. of higher than 1, preferably higher than 2, more preferably higher than 3 and particularly preferably higher than 4, in particularly higher than 5. This distribution coefficient is a measure of the lipophilicity of a substance. As already mentioned above, particularly lipophilic active ingredients profit from the dosage form of this invention. A reason, why such active ingredients can particularly profit, is the fact that normally the resorption of lipophilic active ingredients functions very well, as soon as these substances are dissolved. In other words, the dissolution of these active ingredients from a dosage form is the rate-determining step, when the active ingredient is absorbed into the body.

Nevertheless, there are also active ingredients the lipophilicity of which is such that the formulation proposed here is not sufficient for guaranteeing a sufficient bioavailability. Therefore, the first active ingredient has an n-octanol/water distribution coefficient (log P_{OW}) at 20° C. of preferably at most 100, more preferably at most 50, in particularly at most 30, particularly preferably at most 15 and in particularly at most 7.

For further improving the solubility of the first active ingredient after application, according to the present invention it is preferable to use the first active ingredient in a small particle size. Therefore, the first active ingredient in the dosage form of this invention has preferably mean particle sizes of at most 1000 nm, more preferably at most 600 nm and particularly preferably at most 300 nm According to the present invention, the mean particle size is preferably determined by the analysis method of dynamic light scattering. The small particle size is in particularly important in the case, when the active ingredient does not or hardly dissolve in the emulsifier. This is often the case, when the emulsifier comprises a particularly large hydrophilic part, such as for example at lot of poloxamers, and/or when the first active ingredient is characterized by a particularly high lipophilicity.

According to the present invention it is preferable, when the first active ingredient can be dissolved nearly completely, particularly preferably completely in the emulsifier. So an improved release can be achieved. This effect can be achieved by adjusting the lipophilic part of the emulsifier to the lipophilicity of the active ingredient. How this can be achieved can be followed from the information about the emulsifier and its molecular structure which is given below. Thus particularly preferably, the first active ingredient is present in the dosage form according to the present invention in a completely dissolved state.

In particular active ingredients which in the case of the use of dosage forms with immediate release have to be administered at least three times a day profit from the dosage form according to the present invention. Thus preferably, the first active ingredient is an active ingredient which in the case of an intake of dosage forms with immediate release has to be administered at least three times a day.

The first active ingredient can be selected from active ingredients for the treatment of pains and for pain therapy, for the treatment of diseases of the nervous system, for the treatment of psychiatric diseases, for the treatment of cardiovascular diseases, for the treatment of migraine, for the treatment of diseases of the respiratory tract and the lung, for the treatment of diseases of the gastrointestinal tract and the pancreas, for the defense against infections including for the treatment of fungal diseases, for the treatment of erectile dysfunction, for hormone therapy, for the treatment of allergic reactions and for the treatment of dizziness of any origin.

Particularly preferably, the first active ingredient is selected from active ingredients of the classes of active ingredient antihistamines, antiemetics, antivertiginous drugs and/or calcium channel blockers. In a particularly preferable embodiment the first active ingredient is cinnarizine, a cinnarizine salt and/or a cinnarizine derivate, in particular a pro-drug. It is particularly preferable, when the first active ingredient is cinnarizine.

The proportion of the first active ingredient of the dosage form is preferably at least 1% by weight, more preferably at least 2% by weight, more preferably at least 3% by weight and particularly preferably at least 5% by weight, based on the total mass of the dosage form according to the present invention. A certain proportion of the active ingredient is necessary for achieving the desired pharmacological effect. The proportion of active ingredient of the first active ingredient must not be too high, because the improvement of the dissolution of the active ingredient cannot be achieved for arbitrarily high proportions of active ingredient with sufficient certainty. Therefore, in preferable embodiments the content of active ingredient of the first active ingredient in the dosage form is limited to at most 25% by weight, more preferably at most 18% by weight, more preferably at most 15% by weight and particularly preferably at most 12% by weight, based on the total mass of the dosage form according to the present invention.

Preferably, the first active ingredient is used in the dosage form according to the present invention in an amount of at least 20 mg or particularly preferably at least 30 mg. The amount of the first active ingredient in the dosage form according to the present invention should preferably not exceed a value of 100 mg and particularly preferably 80 mg.

The second active ingredient is characterized by a solubility that strongly deviates from the solubility of the first active ingredient.

Basically, the processing and the provision of active ingredients with solubility properties that deviate from one another are problematic. For example, there is the risk that the active ingredient with better solubility is released too quickly from the dosage form, while the active ingredient with worse solubility is not or only insufficiently released. With the dosage form according to the present invention it is possible to process active ingredients with solubility properties that deviate from one another, even active ingredients with solubility properties that strongly deviate from one another. According to the present invention, solubility properties that strongly deviate from one another means that the second active ingredient at a pH value of 7 and at 22° C. in aqueous solution has a solubility which exceeds the solubility of the first active ingredient at a pH of 7 and 22° C. in aqueous solution in an extent of at least one decimal power. Preferably, the solubility of the second active ingredient at a pH value of 7 and at 22° C. exceeds the solubility of the first active ingredient in an extent of a factor of even 50, especially preferably a factor of even 100, thus two decimal powers. Such active ingredients particularly profit from the formulation according to the present invention, because their processing together with active ingredients with low solubility in hitherto known dosage forms is stretched to its limits.

The second active ingredient is preferably an active ingredient which comprises at least one hydroxyl group, at least one carboxyl group and/or at least one permanent charge, preferably at least one permanent charge.

In particular such second active ingredients with a long residence time in the body expressed in a long half-life of elimination profit from the dosage form according to the present invention. Because especially in the case of such active ingredients it is particularly important to prevent a release which is too quick, such as for example in the case of a longer residence time in the stomach. According to the present invention, a long half-life of elimination is a half-life of elimination of longer than 2 hours, preferably of longer than 2.5 hours, more preferably of at least 3 hours.

Basically, each second active ingredient having the above mentioned solubility can profit from the provision in the dosage form according to the present invention which should be administered in an extended manner over a longer period of time. Examples of active ingredients and classes of active ingredient which can advantageously be used as a second active ingredient in the context of the present invention are:

Medicines for the treatment of pain and for pain therapy with peripherally acting analgesics, centrally acting analgesics and adjuvant non-analgesics. Preferably, those concerned here are the following analgesics and adjuvant substances:

paracetamol, metamizole, celecoxib, parecoxib, tramadol, pethidine, codeine, dihydrocodeine, piritramide, tilidine, morphine, hydromorphone, oxycodone, levomethadone, fentanyl, sufentanil, buprenorphine, pentazocine, naloxone, flupirtine, carbamazepine, metoprolol, metoclopramide, amitriptyline, doxepin, clomipramine, mianserin, maprotiline, triptans such as e.g. naratriptan, rizatriptan, sumatriptan, zolmitriptan, calcium antagonists such as: flunarizine, topiramate, valproic acid, phenytoin, baclofen, other agents such as: botulinum toxin, ergotamine, lisuride, methysergide, pizotifen, oxcarbazepine, gabapentin and lamotrigine, dexamethasone, methylprednisolone, prednisolone, triamcinolone, diazepam, tetrazepam, tizanidine, butylscopolamine and/or combination of tilidine and naloxone.

Drugs for the treatment of the nervous system, alone or in combination, e.g. seizure disorders (in particular clonazepam, diazepam, lorazepam, midazolam, clobazam, phenytoin, clomethiazole, valproic acid, phenobarbital, gabapentin, lamotrigine, oxcarbazepine, pregabalin, topiramate, ethosuximide, levetracetam, mesuximide, primidone, nitrazepam and/or vigabitrine), Parkinson syndrome (in particular levodopa, with benserazide/carbidopa, bromocriptine, cabergoline, dihydroergocriptine, lisuride, pergolide mesylate, pramipexole, ropinirole, apomorphine, biperidene, metixene hydrochloride, trihexphenidyl, entacapone, amantadine, bupidine, selegiline and/or apomorphine), stroke (in particular clopidogrel, dipyridamole, ticlopidine, heparin, phenprocoumon, warfarin, protamine, phytomenadione, nimodipine, paracetamol, tramadol and/or buprenorphine), intracranial pressure (in particular furosemide and/or mannitol), tremor (in particular propranolol, clozapine, alprazolam and/or primidone).

Drugs for the treatment of psychiatric diseases such as anxiety disorders (in particular alprazolam, diazepam, fluoxetine, paroxetine, chlorprothixene, levomepromazine, thioridazine, flupentixol and/or fluspirilene), depressions (in particular imipramine, amitriptyline, desipramine, maprotiline, minaserine, citalopram, fluoxetine, paroxetine, trazodone, moclobemide and/or mirtazapine), psychoses and schizophrenias (in particular sulpiride, promazine, melperone, thioridazine, chlorprothixene, perazine, pimozide, fluphenazine, olanzapine and/or risperidone), sleep disorders (in particular triazolam, brotizolam, oxazepam, flurazepam, nitrazepam, temazepam, zolpidem tartrate, zopiclon, promethazine, chlorprothixene, pipamperone, thioridazine and/or chloral hydrate), conditions of restlessness and disorientation/confusion (in particular alprazolam, oxazepam, doxepin, clomipramine, imipramine, thioridazine and/or perazine), dementia of the Alzheimer type (in particular donepezil, rivastigmine, tacrine, memantine, nimodipine and/or seleginine).

Drugs for the treatment of cardiovascular diseases, such as coronary heart disease/angina pectoris (in particular clopidogrel, ticlopidine, isosorbide dinitrate, isosorbide mononitrate, nitroglycerin, molsidomine, trapidil, metoprolol, bisoprolol, atenolol, acebutolol, carvedilol, nitrendipine, nifedipine, diltiazem, verapamil, benazepril, lisinopril, ramipril, fosinopril and/or enalapril), cardiac infarction and cardiac insufficiency (in particular isosorbide mono- and dinitrate, clopidogrel, ticlopidine, captoprol, ramipril, lisinopril, candesartan, eproartan, irbesatan, losartan, chlortalidone, xipamide, hydrochlorothiazide, furosemide, piretanide, triameterene, digitalis glycosides, carvedilol, metoprolol and/or prazosine), cardiac arrhythmia (in particular ajmaline, quinidine, disopyramide, flecainide, propafenone, propranolol, carvedilol, amiodarone, verapamil and/or diltiazem), hypertension (in particular metoprolol, atenolol, urapidil, clonidine, dihydralazine, chlortalidone, hydrochlorothiazide, furosemide, felodipine, israpidine, lacidipine, diltiazem, captopril, enalapril, fosinopril, lisinopril, ramnipril, verapamil, candesartan, eprosartan, irbesatan, losartan, doxazosine, bunazosine, prazosine, terazosine, moxonidine, dihydralazine and/or minoxidil).

Drugs, alone or in combination, for the treatment of diseases of the respiratory tract and the lung (in particular theophylline, methylprednisolone, flucortone, dexamethasone, montekulast, roxithromycin, erythromycin, azithromycin, ciprofloxacin, clarithromycin, levofloxacin, ofloxacin, doxycycline, ampicillin and sulbactam, amoxicillin, cefuroxime, clindamycin, cefotiam, cefuroxime, ceftazidime, ceftriaxone, piperacillin and/or moxifloxacin).

Drugs for the treatment of erectile dysfunction (in particular sildenafil, tadalafil, vardenafil, theobromine, caffeine and/or theophylline).

Preferably, the second active ingredient is an active ingredient which together with the first active ingredient shows a synergistic effect. According to the present invention this means that the effect of the first and the second active ingredients, when their intake is realized by the dosage form according to the present invention, is increased in comparison to the single use of the first or the second active ingredients. Especially in the case of such synergistically effective active ingredients it is important that the release profiles of both active ingredients are or become adjusted to one another. Only in such a manner it can be achieved that the synergistic effect maintains during the whole time. Thus, especially for synergistically active ingredients this invention provides a considerable improvement compared to prior art. It is especially preferable, when the first and also the second active ingredients are of the same class of active ingredient.

It is particularly preferable, when the second active ingredient is selected from the class of active ingredient antihistamines, antiemetics, calcium channel blockers and/or antivertiginous drugs. It is especially preferable, when the second active ingredient is dimenhydrinate.

Preferably, the second active ingredient is contained in the dosage form according to the present invention in the form of small particles. This means that the mean particle size of the second active ingredient does preferably not exceed 1000 nm More preferably, the mean particle size of the second active ingredient is not higher than 300 nm. The particle size of the active ingredient is determined by means of laser diffraction.

The proportion of the second active ingredient of the dosage form is preferably at least 4% by weight, more preferably at least 8% by weight, based on the total mass of the dosage form according to the present invention. The proportion of active ingredient of the second active ingredient must not be too high, because the modification of the release cannot be achieved for arbitrarily high proportions of active ingredient with sufficient certainty. Therefore, in preferable embodiments the content of active ingredient of the second active ingredient in the dosage form is limited to at most 30% by weight, more preferably at most 18% by weight.

In preferable embodiments the dosage form according to the present invention does not comprise antibiotics in pharmaceutically active amounts, in particular no representative of the class of active ingredient tetracyclines. Antibiotics compromise the flora of the intestine by killing important bacteria in the intestine. In the case of the dosage form of this invention this effect would result in strong adverse effects, since the dosage form is partially dissolved only very late.

In preferable embodiments the dosage form according to the present invention does not comprise proton pump inhibitors. Proton pump inhibitors increase the pH value in the stomach and thus disturb the dissolution of many drugs, in particular of weakly basic active ingredients.

In preferable embodiments the dosage form according to the present invention does not comprise non-steroidal antirheumatics (NSAR), thus, the first and the second active ingredients as well as optionally the further active ingredients are preferably not selected from active ingredients of the group of non-steroidal antirheumatics. Non-steroidal antirheumatics may result in an increase of the production of gastric acid and in a reduction of the formation of gastric mucus which may be accompanied by an impairment of the gastric and intestinal mucosa. This may disadvantageously influence the resorption of the proportion of the active ingredient contained in the dosage form.

Besides the first and the second active ingredients, in the dosage form according to the present invention further active ingredients may be contained. But especially preferably, the proportion of active ingredient consists of the first and the second active ingredients.

In this case the total content of active ingredient of the dosage form, preferably consisting of the first and the second active ingredients, is preferably at most 50% by weight, more preferably at most 30% by weight and particularly preferably at most 28% by weight, based on the total mass of the dosage form according to the present invention. When the total content of active ingredient of the dosage form is too high, then it is possible that this does not longer optimally result in the increase of the dissolution and/or the modification of the release of the active ingredients, and/or that the dosage form in total, for guaranteeing an optimal release, would become too large and thus the swallowability would become worse.

The first and/or the second active ingredients may have an unpleasant taste, such as for example a bitter and/or anesthetic taste. In particular such first and/or second active ingredients profit from the dosage forms according to the present invention. Namely, the composition of the dosage form according to the present invention allows an optimal masking of an unpleasant taste of the first and/or the second active ingredients. And it may also be the case that the first and/or the second active ingredients are characterized by limited storage stability. With the dosage forms according to the present invention also active ingredients with limited storage stability can be optimally provided in a storage-stable form.

The emulsifier improves the dissolution of the first active ingredient and at the same time is conducive to the modification of the release of the first and the second active ingredients. This is particularly important so that at least a part of the first active ingredient is already dissolved in the stomach in an as quick as and also as uniform as possible manner. As described above, the pH value of the stomach after the ingestion of a meal decreases only slowly again. But it is advantageous that already now the first active ingredient is dissolved in the stomach so that it is mixed with the chyme. The mixing is effected by the motion of the stomach.

Preferably, the first active ingredient is embedded in an emulsifier matrix, and it is particularly preferably dissolved therein. So the surface of the first active ingredient is enlarged. At the same time, the emulsifier preferably only dissolves slowly in the gastrointestinal tract, and thus it is capable of modifying the release of the first and the second active ingredients.

So that the improvement of the dissolution and the modification of the release of the proportion of active ingredient are particularly well-marked, the emulsifier should preferably have an HLB value of at least 1 and at most 16, in particular at least 9 and at most 14. Preferably, the emulsifier is non-ionic.

It was shown that such emulsifiers which have a certain structural motif are capable of achieving particularly advantageous results. This structural motif is a polyethylene glycol chain. It was found that emulsifiers with this structural motif, thus with at least one polyethylene glycol chain, have a dual function. On the one hand, at high pH values of about pH 4 or higher they result in an improvement of the solubility of the first active ingredient. On the other hand, at low pH values, namely then when the first active ingredient preferably has a higher solubility, they result in a delay of the release. This is a behavior which is desirable according to the present invention, because the release of the first active ingredient should occur as continuously as possible over a long period of time.

The polyethylene glycol chain is the hydrophilic part of the emulsifier and is connected with the lipophilic part. Advantageously, the lipophilic part may be a glyceride group or another polyalkylene oxide chain.

As glyceride groups mono- and diglyceride groups can be used, wherein diglyceride groups are preferable. Depending on the chain length of the used fatty acid groups monoglyceride groups may optionally be not lipophilic enough.

As polyalkylene oxide chains in particular polyalkylene oxides with uninterrupted carbon chains of at least 3 carbon atoms may be used. A preferable example of a polyalkylene oxide chain in the emulsifier of this invention is polypropylene oxide.

These emulsifiers can be obtained according to methods which are known by a person skilled in the art. Polyethylene glycol glycerides can for example be prepared by reaction of the respective glycerides with polyethylene glycol. Commercially available polyethylene glycol glycerides are for example Gelucire® 43/01 and Gelucire® 50/13.

In other words: the emulsifiers which are used in the dosage form according to the present invention comprise the following structural element:

R¹—O—[CH₂—CH₂—O]ₙ—R² (formula I)

In this formula R¹ and R² are the same or different. R¹ and R² independently from each other are hydrogen, alkyl, glyceride or polyalkylene oxide. Alkyl is preferably a short-chain alkyl, i.e. its chain length amounts to at most 6 carbon atoms. Preferably, R¹ and R² independently from each other are hydrogen, glyceride or polyalkylene oxide.

When one group R¹ or R² is hydrogen, then the groups R¹ and R² are different, i.e. the other group each is not hydrogen; and when R¹ or R² is alkyl, then the groups R¹ and R² are different, i.e. the other group each is not alkyl. Otherwise, the molecule would not have the required emulsifying effect. Preferably, R¹ and R² are neither hydrogen nor alkyl, when the other group each is hydrogen or alkyl.

n is the number of the chain members in the polyethylene oxide chain. n is an integer of at least 4, preferably at least 10 and particularly preferably at least 20. When n is lower, then the desired effect according to the present invention is not very strong, because the lipophilic part of the emulsifier is too small. In preferable embodiments n is not higher than 100, in particular not higher than 50, more preferably not higher than 40 or not higher than 30. It was shown that longer chains result in slower degradation of the emulsifier by pancreatin and lipases. With that the extended release can be controlled. But when the chain is too long, then the dosage form does not release the first active ingredient in a sufficient rate.

When R¹ and/or R² are a polyalkylene oxide group, then the moiety has the following general formula:

—O—[Y—O]ₘ—R³ (formula II)

In this formula R³ is hydrogen or alkyl, in particular short-chain alkyl (up to C₆). Y is an alkylene group having a carbon chain length of at least C₃ and preferably at most C₆, more preferably at most C₄.

m is the number of the chain members in the polyalkylene oxide chain. m is preferably an integer of at least 3, in particular at least 5 and particularly preferably at least 10. Preferably, m should not exceed an integer of 50, in particular 40 and particularly preferably 30 or 20. In preferable embodiments R¹ and R² are polyalkylene oxide groups.

When R¹ and/or R² are glyceride groups, then the glyceride group has the following general formula:

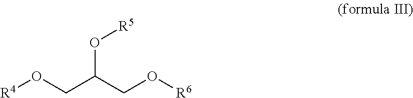

In this case formula III at one of the positions R⁴, R⁵ or R⁶ is connected with the structural element of formula I. According to the present invention it is irrelevant at which position the structure of formula III is connected with the structure of formula I. Thus one of the groups R⁴, R⁵ or R⁶ is the structure of formula I. For the remaining two groups the following is true:

R⁴ is preferably hydrogen or a fatty acid group. The fatty acid group R⁴ has a chain length of preferably at least C₆, more preferably at least C₈ and particularly preferably at least C₁₀. A minimum chain length should be fulfilled, so that the lipophilic property of the glyceride group is sufficient. The number of the carbon atoms in the fatty acids is preferably even. Preferably, the chain length should not exceed a value of C₂₂, more preferably C₁₈ and particularly preferably C₁₆, because otherwise the solubility of the emulsifier may be compromised.

R⁵ is preferably hydrogen or a fatty acid group. The fatty acid group R⁵ has a chain length of preferably at least C₆, more preferably at least C₈ and particularly preferably at least C₁₀. A minimum chain length should be fulfilled, so that the lipophilic property of the glyceride group is sufficient. The number of the carbon atoms in the fatty acids is preferably even. Preferably, the chain length should not exceed a value of C₂₂, more preferably C₁₈ and particularly preferably C₁₆, because otherwise the solubility of the emulsifier may be compromised.

R⁶ is preferably hydrogen or a fatty acid group. The fatty acid group R⁶ has a chain length of preferably at least C₆, more preferably at least C₈ and particularly preferably at least C₁₀. A minimum chain length should be fulfilled, so that the lipophilic property of the glyceride group is sufficient. The number of the carbon atoms in the fatty acids is preferably even. Preferably, the chain length should not exceed a value of C₂₂, more preferably C₁₈ and particularly preferably C₁₆, because otherwise the solubility of the emulsifier may be compromised.

Preferably, two of the groups R⁴, R⁵ and R⁶ are fatty acid groups. So then it is a diglyceride group.

It has to be considered that the correct chain length of the fatty acid groups strongly corresponds with the number of the chain members in the polyethylene oxide chain (n). When the polyethylene oxide chain is longer, then also the fatty acid chains may be longer.

The emulsifiers described here can be produced by reaction of respective mono-, di- and/or triglycerides with respective polyalkylene oxides. These educts are commercially available. The emulsifiers according to the present invention are preferably mixtures of the mentioned substances.

In preferable embodiments the emulsifier is selected from Gelucire® 50/13, Gelucire® 43/01, Poloxamer 407 and mixtures thereof, wherein Gelucire® 50/13 is especially preferably.

Good solubilization is achieved, when the ratio of the masses of first active ingredient to emulsifier is at least 1 to 30, more preferably at least 1 to 20 and particularly preferably at least 1 to 10. The mentioned ratio is preferably at most 1 to 1, more preferably at most 1 to 2 and particularly preferably at most 1 to 3. Basically it is true, that a higher amount of emulsifier enables better solubilization. In addition, the emulsifier preferably also is conducive to the modification of the release of the second active ingredient. But it has to be considered that a dosage form should not exceed certain maximum volumes, so that the intake thereof is not unnecessarily unpleasant. In addition, a too high amount of emulsifier may also result in hindrance of the dissolution and/or the release of the first active ingredient.

A mass ratio of the second active ingredient to the emulsifier of at least 1:20, preferably at least 1:10 and particularly preferably at least 1:5 is advantageous, because the emulsifier should also be conducive to the modification of the second active ingredient. When the proportions of the emulsifier in relation to the second active ingredient are too high, then there is the risk that the release of the second active ingredient is hindered and that the dosage form in total becomes too large. But the mass ratio of second active ingredient to emulsifier should exceed a value of 1:1.2 and preferably 1:1.5. When the proportion of the emulsifier in relation to the second active ingredient is too low, then the emulsifier cannot be sufficiently conducive to the modification of the release of the second active ingredient.

The dosage form contains the emulsifier preferably in a proportion of at least 15% by weight, more preferably at least 20% by weight, still more preferably at least 22% by weight and particularly preferably at least 25% by weight, based on the total mass of the dosage form. The proportion of the emulsifier should preferably not exceed a proportion of 50% by weight, more preferably 40% by weight and particularly preferably 35% by weight, based on the total mass of the dosage form. In tests for determining the optimum ratio of solubilization and volume of the dosage form it was shown that these amounts are advantageous.

In particularly preferable embodiments instead of the above mentioned substances or in addition to the above mentioned substances at least one phospholipid is used. The phospholipid is preferably selected from phosphatidylcholines (lecithins), phosphatidylethanolamines (kephalins), phosphatidylserines, sphingomyelins and mixtures thereof. Especially preferable are phosphatidylcholines (lecithins). Surprisingly, it was found that phospholipids also result in the above-described advantageous effects, like the above mentioned emulsifiers.

Thus, the emulsifier may be a phospholipid. Preferably, the emulsifier comprises the phospholipid in addition to at least one of the above mentioned substances. In such a case the phospholipid strengthens the advantageous effects of the above mentioned substances.

The dosage form of this invention comprises at least one controlled-release agent. This serves for the modification of the release of at least the second active ingredient, wherein it is more preferable, when the controlled-release agent has no measurable influence onto the release of the first active ingredient.

Suitable controlled-release agents may be of inorganic or organic origin. Preferably, the controlled-release agent is selected from a natural polymer, a synthetic polymer and mixtures thereof.

It is particularly preferable, when the controlled-release agent is swellable in water, thus is capable of increasing its volume by absorption of water and thus of providing a diffusion barrier for the second active ingredient. Preferable controlled-release agents are characterized by a swelling capability of higher than 1.5, preferably higher than 3, more preferably higher than 5. It is advantageous, when the controlled-release agent has a swelling capability of at most 20, preferably at most 15. This means that the volume of the swollen controlled-release agent is preferably not larger than the twentyfold, preferably not larger than the fifteenfold of the volume of the non-swollen controlled-release agent. A swelling capability of the controlled-release agent which is too high may hinder the release of the second active ingredient, since it is preferably contained in the dosage form in a relatively high proportion.

The swelling capability can be determined by methods which are known by a person skilled in the art, such as for example by microscopic monitoring of the increase of volume, when swelling occurs under suitable conditions for the initiation of swelling each.

Preferably a controlled-release agent is used which in an aqueous solution in a proportion of 2% by weight at a temperature of 20° C. and a pressure of 101.325 kPa results in a viscosity of at least 500 mPas. The viscosity is measured with a capillary viscometer (DIN 53015). According to the desired extension of the release of the second active ingredient is may be desired, also to choose higher viscosities. The higher the viscosity of the controlled-release agent, the stronger is the modification of the release. In especially preferable embodiments the viscosity of the controlled-release agent is even at least 2500 mPas and particularly preferably at least 3500 mPas. But when the viscosity is too high, then the release of the second active ingredient is too slow. This would have the result that optionally the second active ingredient during its passage through the gastrointestinal tract is not completely released or only in lower intestine sections, so that it would not be possible to achieve therapeutic plasma levels. Preferably, the viscosity is limited to a value of at most 200.000 mPas, more preferably of at most 120.000 mPas, still more preferably of at most 90.000 mPas and most preferably of at most 15.000 mPas.

Preferable controlled-release agents have a mean molecular weight (number average MN) of at least 5.000, more preferably at least 12.000 and particularly preferably at least 20.000. Controlled-release agents with mean molecular weights which are too low often are not characterized by the required strengths for preparing stable and abrasion-resistant dosage forms. On the other hand, controlled-release agents with mean molecular weights which are too high often result in worse dissolution so that the release of the second active ingredient may be hindered and there is the risk that it is released only in low intestine sections and thus cannot be sufficiently resorbed any longer. Preferably, the controlled-release agent has a mean molecular weight of at most 250.000, more preferably at most 200.000 and most preferably at most 150.000.

Preferably, the controlled-release agent is selected from natural or synthetic polysaccharides, polymethacrylates and their copolymers, polyacrylates and mixtures thereof.

It is particularly preferable, when the controlled-release agent comprises a natural or synthetic polysaccharide. Particularly preferably, the controlled-release agent consists of a natural or synthetic polysaccharide. Preferable polysaccharides comprise more than 10 monosaccharide units. It was found that pentoses and hexoses, more preferably selected from glucose, galactose, xylose, fructose, arabinose, mannose, mannuronic acid, guluronic acid, gulose and mixtures thereof are suitable monosaccharide units.

Particularly preferable natural or synthetic polysaccharides are selected from celluloses, cellulose derivatives, alginates and mixtures thereof.

It is particularly preferable, when the controlled-release agent comprises a cellulose derivative. Suitable cellulose derivatives have mean degrees of substitution of at least 1, more preferably at least 1.2 and most preferably at least 1.3. The degree of substitution is the mean number of substituents per glucose unit of the cellulose. When the degree of substitution is too low, then the swelling capability of the controlled-release agent and the viscosity may be reduced so that the release of the second active ingredient is not sufficiently modified any longer.

Preferable cellulose derivatives are selected from methylcellulose, hydroxypropylcellulose, hydroxypropylmethylcellulose and mixtures thereof. Particularly preferable cellulose derivatives are selected from methylcellulose, hydroxypropylmethylcellulose and mixtures thereof. A most preferable cellulose derivative is hydroxypropylmethylcellulose. It is especially preferable, when the controlled-release agent consists of hydroxypropylmethylcellulose.

It was shown that highly viscous hydroxypropylmethylcelluloses having a viscosity of higher than 1.000 mPas are particularly suitable as controlled-release agent. Preferably, the viscosity of the hydroxypropylmethylcellulose is not higher than 120.000 mPas, more preferably not higher than 20.000 mPas. These values have been shown to be particularly advantageous according to the present invention for optimally modifying the release of the second active ingredient. The determination of the viscosity is conducted according to the above-described methods and measuring conditions.

Particularly preferably, the controlled-release agent does not comprise polymers with permanent charge. Particularly preferably, the controlled-release agent generally does not comprise ionic substances, in particular no sodium carboxymethyl celluloses. According to the present invention this means that the controlled-release agent comprises substances with permanent charge only in an amount of less than 15% by weight, preferably less than 5% by weight and more preferably at most 1% by weight. It is especially preferable, when no substances with permanent charge are contained in the controlled-release agent. Because, when substances with permanent charge are contained in the controlled-release agent in too high amounts, there is the risk that this results in the formation of a precipitate with other substances with permanent charge, such as for example the second active ingredient. The formation of a precipitate may be disadvantageous for the stability of the dosage form and/or the release of the second active ingredient. Thus it is particularly preferable, when the controlled-release agent and more preferable the dosage form according to the present invention do not comprise any sodium carboxymethyl celluloses.

The proportion of the controlled-release agent of the dosage form should preferably be at least 15% by weight, more preferably at least 20% by weight, still more preferably at least 22% by weight and particularly preferably at least 25% by weight. When an amount of the controlled-release agent is used which is too low, then the desired effect cannot be achieved. When the amounts are too high, then the release of the second active ingredient is not quick enough and the processability of the dosage form may considerably be hindered. Therefore, the proportion of the controlled-release agent of the dosage form should not exceed a value of 55% by weight, more preferably 45% by weight and particularly preferably 35% by weight.

An essential feature of the dosage form according to the present invention is that the sum of the proportions by weight of the emulsifier and the controlled-release agent is at least 40% by weight, based on the total mass of the dosage form. Both, the emulsifier and also the controlled-release agent are conducive to the modification of the release of the second active ingredient. Therefore it is important that the sum of the proportions is at least 40% by weight, for optimally modifying the release of the second active ingredient and for effectively preventing peaks of plasma levels through a too quick release, in particular in the case of longer residence times of the dosage form in the stomach. Especially the synergistic coaction of emulsifier and controlled-release agent allows here an optimal release of the second active ingredient together with optimal processability of the components of the dosage form. As described above, too high proportions of emulsifier as well as too high proportions of controlled-release agent of the dosage form are disadvantageous for the processability and the release of the first and/or the second active ingredients. Therefore, it was shown that it is particularly advantageous to use a combination of emulsifier and controlled-release agent which comprises at least 40% by weight of the total mass of the dosage form. More preferably, the sum of the proportions by weight of emulsifier and controlled-release agent of the dosage form according to the present invention is at least 45%, still more preferably at least 48% and especially preferably at least 52%.

Thereby it was shown that a mass ratio of emulsifier to controlled-release agent of at least 1:10, preferably at least 1:5 and still more preferably at least 1:2 as well as particularly preferably at least 1:1.2 is advantageous. The mass ratio is preferably at most 10:1, more preferably at most 5:1, still more preferably at most 2:1 and particularly preferably at most 1.2:1. In especially preferable embodiments of the dosage form according to the present invention the mass ratio of emulsifier to controlled-release agent is 1:1, wherein in these embodiments the emulsifier and the controlled-release agent are contained in the dosage form according to the present invention in a proportion of at least 20% by weight each.

According to the present invention the mass ratio of the second active ingredient to the controlled-release agent is lower than 1:1, more preferably at most 1:1.2, still more preferably at most 1:1.5 and particularly preferably at most 1:1.8. When the proportion of the controlled-release agent relative to the second active ingredient is too low, then the release of the second active ingredient is not sufficiently modified. Then there is the risk that already in the stomach too much second active ingredient is released, with the consequence of toxic plasma levels. But the mass ratio should not fall below a value of 1:20, more preferably 1:15, still more preferably 1:10 and particularly preferably 1:4. When the proportions of the amount of controlled-release agent are too high, then the release of the second active ingredient is delayed in a too large extent and it may optionally be released only in low intestine sections. Then only insufficient plasma levels are achieved. In addition, the dosage form in total becomes too large and thus the swallowability becomes worse.

Optionally, the dosage form may comprise at least one binder. The binder serves for binding the other components of the dosage form according to the present invention to one another, being also called “bonding capacity” of the binder. In such a case the binder is in particularly suitable for binding the second active ingredient and the controlled-release agent to one another.

The binders which are used according to the present invention have the advantage that they can be processed with the other components of the dosage forms according to the present invention very easily and that they already in low amounts result in an excellent effect. In addition, the binders facilitate the processing of the components of the dosage form according to the present invention and they are conducive to stabilize the dosage forms in total, preferably to increase the mechanical stability of the dosage forms during production. For example, the binders are suitable for increasing the melting point of a mixture of first active ingredient and emulsifier during the production of the dosage forms according to the present invention so that further treatment of this mixture can easily be conducted.

Suitable binders may be of inorganic or organic origin. Preferably, the binder is a natural or synthetic polysaccharide comprising two or more identical or different monosaccharide units, particularly preferably selected from glucose, galactose and mixtures thereof. Such substances are available very cheaply, can be processed very easily and at the same time partially show a certain disintegrating effect. It is particularly advantageous, when the binder at the same time is suitable for masking a bad taste of the first and/or the second active ingredients. Therefore, preferable binders have a sweet taste.

It is particularly preferable, when the binder is selected from lactose, starch, starch derivatives and mixtures thereof. From the starches corn starch is particularly preferable due to the easy processing and advantageous properties. Especially preferably the binder is selected from lactose, corn starch and mixtures thereof, wherein more preferably the binder consists of lactose, corn starch or mixtures thereof.

The proportion of this binder of the dosage form should be preferably at least 5% by weight, more preferably at least 10% by weight and particularly preferably at least 12% by weight. Preferably, the proportion of binder of the dosage form should not exceed a value of 40% by weight, more preferably 30% by weight and particularly preferably 20% by weight. An amount of binder which is too high increases the size of the dosage form and is therefore not desired.

But there are embodiments of the invention, in which no binder is used. Then the dosage forms according to the present invention can be obtained with very good stability in a still quicker and cheaper manner.

The dosage forms according to the present invention may comprise lubricants, in particular in the case, when compression of the components of the dosage forms according to the present invention is desired.

Lubricants according to the present invention are suitable for further facilitating the production of the dosage forms, which according to the present invention is called “lubricating effect”. In preferable embodiments in which the dosage form according to the present invention is designed as a tablet the lubricants facilitate for example the compression of the other components of the dosage form into a tablet.

According to the present invention, lubricants are selected from flow regulating agents, lubricating agents, mold release agents and mixtures thereof.

Preferably, the lubricant is selected from stearic acid, stearates, calcium behenate, hydrogenated vegetable fats, talcum, highly disperse silica, polyethylene glycols, sodium dodecyl sulfate, magnesium dodecyl sulfate and mixtures thereof. Suitable stearates comprise for example magnesium stearate, glycerin monostearate, glyceryl palmitostearate or mixtures thereof.

Preferable lubricants are lipophilic. According to the present invention, the lubricants are preferably virtually not soluble in aqueous solution at 22° C. and a pH value of 7. This results in the advantage that the lubricants in particular can optimally reduce the adherence of the further components of the dosage form during their processing at machines and/or apparatuses which are used for that as well as the interparticle friction. Therefore, the lubricant is more preferably selected from stearic acid, stearates, calcium behenate, hydrogenated vegetable fats, talcum, highly disperse silica and mixtures thereof.

It was shown that it is particularly advantageous, when the lubricant is selected from magnesium stearate, talcum, highly disperse silica and mixtures thereof. It is especially preferable, when a mixture of all three components is used as lubricant, wherein this mixture is also called “lubricant mixture”. This results in the advantage that the properties of all three components can be used in an advantageous manner for the dosage forms according to the present invention and that the effects of the components complement and/or potentiate each other in an optimal manner.

But here it has to be considered that highly disperse silica is not contained in the lubricant mixture in a too high amount. Amounts of highly disperse silica which are too high can disadvantageously influence and reduce the lubricating effect of magnesium stearate. Therefore, the content of highly disperse silica of the lubricant mixture has to be limited to a value of preferably 25% by weight, more preferably 20% by weight and particularly preferably 18% by weight.

Preferably, the proportion of magnesium stearate in relation to the proportion of talcum in the lubricant mixture should be low. Preferably, the mass ratio of magnesium stearate to talcum should be at most 1:1.3, more preferably at most 1:1.5, still more preferably at most 1:1.8. When the proportion of magnesium stearate in relation to the proportion of talcum was higher, then, with concurrent presence of talcum, an impairment of the lubricating effect of magnesium stearate was observed.

A preferable lubricant mixture comprises:
between 5% by weight and 25% by weight of highly disperse silica, more preferably between 8% by weight and 17% by weight; and
between 20% by weight and 40% by weight of magnesium stearate, more preferably between 22% by weight and 35% by weight; and
between 45% by weight and 75% by weight of talcum, more preferably between 50% by weight and 65% by weight.

The proportion of lubricant in the dosage forms according to the present invention is preferably limited to at most 15% by weight, more preferably at most 12% by weight and more preferably at most 10% by weight. A content of lubricant which is too high may promote segregation during the production of the dosage form so that the excellent uniformity of the content of the dosage form according to the present invention is no longer guaranteed. In addition, the wettability of the dosage forms according to the present invention and thus their disintegration may be influenced in a disadvantageous manner.

In preferable embodiments in which a lubricant is contained in the dosage form according to the present invention the minimum content of lubricant is preferably at least 1% by weight, more preferably at least 2% by weight and particularly preferably at least 4% by weight, based on the total mass of the dosage form according to the present invention.

Optionally, the dosage forms according to the present invention may further contain additional adjuvants. Such adjuvants may be selected from fillers, further binders, surfactants, fatty alcohols, triglycerides, antioxidants, disintegrants, complexing agents, coating agents, preservatives, softening agents, pigments and mixtures of such substances. A person skilled in the art is capable of choosing accordingly pharmaceutically acceptable adjuvants. In such a case the proportion of optional other adjuvants of the dosage form according to the present invention is preferably at most 10% by weight, more preferably at most 8% by weight and particularly preferably at most 5% by weight, based on the total mass of the dosage form.

In particularly preferable embodiments of the dosage form according to the present invention no additional adjuvants are contained in the dosage form, thus the dosage form according to the present invention consists of first and second active ingredients, emulsifier, controlled-release agent, optionally binder and optionally lubricant.

In summary, the dosage form may inter alia contain:
a. a first active ingredient (preferably in a proportion of at least 1% by weight and at most 25% by weight, based on the total mass of the dosage form);
b. a second active ingredient (preferably in a proportion of at least 4% by weight and at most 30% by weight, based on the total mass of the dosage form);
c. an emulsifier for improving the dissolution of the first active ingredient and for modifying the release of the first and the second active ingredients (preferably in a proportion of at least 15% by weight and at most 50% by weight, based on the total mass of the dosage form), in particular for improving the solubility of the first active ingredient at high pH values and for decelerating the dissolution of the first active ingredient at low pH values;
d. a controlled-release agent for modifying the release of the second active ingredient (preferably in a proportion of at least 15% by weight and at most 55% by weight, based on the total mass of the dosage form), in particular for decelerating the dissolution of the second active ingredient at low pH values;
e. optionally a binder for supporting the processing of the other components of the dosage form according to the present invention during the production and for optimizing the mechanical stability (preferably in a proportion of at least 5% by weight and at most 40% by weight, based on the total mass of the dosage form);
f. optionally a lubricant for improving the processability of the other components of the dosage form according to the present invention, in particular the compression of the other components (preferably in a proportion of at least 1% by weight and at most 15% by weight, based on the total mass of the dosage form);
g. optionally other adjuvants;
h. optionally further active ingredients;

wherein the sum of the proportions by weight of emulsifier and controlled-release agent is at least 40% by weight, based on the dosage form.

In prior art no convincing dosage form is provided which allows a modified release each in one dosage form of an active ingredient with low solubility (here: first active ingredient) and also of a second active ingredient with better solubility. Because the release of the active ingredient with low solubility has to be promoted and at the same time the release of the active ingredient with better solubility has to be suppressed. This invention provides a solution for this problem by providing a dosage form according to the present invention comprising both active ingredients together with at least one emulsifier and at least one controlled-release agent.

In the context of the dosage form described here it was a challenge to delay the release of the second active ingredient. One reason for that is that for solubilizing of the first active ingredient a relatively high amount of emulsifier is required. This results in the fact that the volume of the dosage form is already nearly exhausted to the utmost by the required measure with respect to the improvement of the solubility of the first active ingredient. Thus, the measure with respect to the delay of the release of the second active ingredient must be possible in a volume which is as small as possible. This becomes possible by containing a controlled-release agent for modifying the release of the second active ingredient and by selecting the emulsifier such that it at the same time also is conducive to the modification of the release of the second active ingredient. For that proportions of emulsifier and controlled-release agent are required which together comprise at least 40% by weight of the dosage form.

A coating over the whole dosage form is not preferable, because this coating might disadvantageously influence and in particularly disadvantageously delay the release of both active ingredients. Thus according to the present invention it is preferable that the dosage form does not comprise any coating, in particular a coating which dissolves delayed in a time-dependent manner and/or dependent on the pH value. Thus, the dosage form according to the present invention particularly does not comprise a coating comprising cellulose derivatives, methacrylic acid polymers, polyvinyl derivatives, shellac and mixtures thereof.

A particularly preferable embodiment of the dosage form according to the present invention comprises as the first active ingredient cinnarizine and as the second active ingredient dimenhydrinate. The dosage form is suitable for use in therapy of dizziness of any origin. Therefore, also the use of this dosage form for the treatment of dizziness of any origin is according to the present invention. Also a method of administering the dosage form according to the present invention to a patient with a need for a treatment with one or both of the active ingredients contained in the dosage form, in particular to a patient suffering from dizziness regardless of which origin is according to the present invention.

The dosage forms of the invention are characterized by the advantage that they are particularly storage-stable, which means that the criteria of storage stability according to ICH are at least fulfilled and preferably exceeded, i.e. the dosage forms according to the present invention show better values than would be sufficient. Storage stability means that during a storage at certain storage conditions and a certain storage time which follow from the ICH guideline Q3B (R2) (Impurities in New Drug Products) a sufficiently high content of drug of higher than 90%, based on the original amount of active ingredient, is available and that degradation products which might be a hazard for the patients do not exceed a certain maximum. The maximums follow from the ICH guideline Q3B (R2). In a short-term stress test at 40° C. and 75% relative humidity according to the ICH guideline Q3B (R2) over 6 months the dosage forms according to the present invention showed very good storage stability. After the storage the dosage forms according to the present invention still contained more than 90% of the active ingredient, based on the amount of active ingredient in the dosage form which has not been stored, and the amount of impurities was within the acceptable limits according to the ICH guideline Q3B (R2). The dosage forms according to the present invention are preferably storage-stable over a storage time of longer than 6 months, preferably at least 12 months under standard conditions according to international guidelines of ICH.

The dosage forms according to the present invention are characterized by an excellent uniformity of the mass and uniformity of the content, which are guaranteed by the composition of the dosage form and the production method. The tests are conducted according to the respective methods of the European Pharmacopoeia (Ph. Eur. 7). A dosage form shows preferably a uniformity of the mass such that the mass of 20 such dosage forms does preferably only deviate from the average mass of the dosage form which follows from the mass of the 20 dosage forms in an extent of lower than 5%, more preferably lower than 4%. The dosage form according to the present invention shows preferably a uniformity of the content such that the content of active ingredient of 10 such dosage forms each is between 85% and 115%, preferably between 87% and 113% and ideally between 90% and 110%, based on the average content of the active ingredient of the 10 dosage forms.

Preferably, the dosage form according to the present invention is designed as a tablet, wherein this tablet is more preferably characterized by excellent mechanical stability. As a measure for the mechanical stability of the dosage forms according to the present invention the fracture strength and the friability can be used.

Preferably, the tablet has a fracture strength of at least 40 N, more preferably at least 50 N. But preferably, the fracture strength should not exceed a value of 200 N, preferably 150 N, more preferably 130 N and particularly preferably 100 N. When the fracture strength is too high, then the disintegration of the tablet is measurably worse. The fracture strength of a tablet is determined according to a respective standard method of the European Pharmacopoeia with an apparatus consisting of two jaws (Ph. Eur. 7), wherein the test is conducted with 10 tablets.

Preferably, the loss of mass of the dosage form according to the present invention in the friability test according to the European Pharmacopoeia (Ph. Eur. 7), conducted with at least 10 dosage forms, is lower than 1%, preferably even lower than 0.8% and more preferably lower than 0.6%. Thus, the requirements of the European Pharmacopoeia with respect to the friability are preferably exceeded.

Furthermore, the dosage forms according to the present invention have the advantage that they can be used in a flexible manner. So it is possible to provide the dosage form according to the present invention, which is present as a tablet, with at least one additional layer containing active ingredient, which according to the present invention is called “additional layer”.

In embodiments of the present invention, therefore, the dosage form according to the present invention is a tablet which can be provided with at least one additional layer, preferably exactly one additional layer. The additional layer can release at least one active ingredient in modified form or immediately. In this case “immediate release” means preferably, that after 60 minutes at least 65% of the active ingredient, preferably at least 75% and more preferably at least 80% of the active ingredient are released into the test medium, preferably gastric juice. The measurement can be conducted according to common methods of release measurements, in particular according to the methods of the European Pharmacopoeia or the American Pharmacopoeia (USP), preferably with the Bio-Dis apparatus (200 ml test medium, 25 dpm, 37° C.). The exact conditions depend on the respective active ingredient and the dosage form and can be found in well-established pharmacopoeias.

In the embodiments in which the dosage form is provided with an additional layer it is preferable, that the additional layer does only cover a part of the dosage form, so that the release of the first and the second active ingredients from the dosage form is not hindered. Furthermore, there is the risk that the swallowability is worse, when the additional layer completely encloses the dosage form, thus the total surface of the dosage form is completely covered with the additional layer. It is preferable, when the additional layer covers the total surface of the dosage form in an extent of at most 70%, more preferably at most 65% and particularly preferably at most 50%.

Furthermore, the additional layer should not have a too high mass, so that the dosage form and the additional layer together can still be swallowed well. Therefore, the mass of the additional layer is at most 400 mg, preferably at most 350 mg and particularly preferably at most 300 mg. But a certain minimum mass is required so that the additional layer can be bonded to the dosage form. Therefore, the mass of the additional layer is preferably at least 100 mg, more preferably at least 150 mg.

It is advantageous, when the mass ratio of the additional layer to the dosage form is at most 1:1.5, preferably at most 1:1.8 and more preferably at most 1:2. When these ratios were fulfilled, then good swallowability was given.

The additional layer may comprise the first and/or the second active ingredients. It is advantageous, when the additional layer contains the first and the second active ingredients and immediately releases both active ingredients. This supplements the modified release of the first and the second active ingredients from the dosage form.

For example in the case of long-term therapies with certain active ingredients it may be advantageous to achieve a therapeutic plasma level within a short period of time, in particular, when in long dosage intervals at the end of the interval the concentration falls below the minimum effective concentration. Also in the case of short-term therapies it can be desired to achieve a therapeutic plasma level within a short period of time. With the dosage form according to the present invention which releases the first and the second active ingredients in a modified manner by the downstream release the effect is subsequently extended.

When the first and the second active ingredients are contained in the additional layer, then it was shown that it is advantageous, when the mass ratio of the first active ingredient in the dosage form to the first active ingredient in the additional layer and/or the mass ratio of the second active ingredient in the dosage form to the second active ingredient in the additional layer are between 1:1 and 2.5:1. Such a distribution of active ingredient is conducive to achieve an optimal profile of the plasma level, in particular in the case of immediate release from the additional layer, without any toxic plasma levels and undesired fluctuations of the plasma level.

When the proportion of active ingredient in the additional layer is too high, then it may be possible that in particular in the case of immediate release from the additional layer too high plasma levels of first and/or second active ingredients and/or a too steep increase of the plasma levels result. Preferably, the amount of the first and/or the second active ingredients in the additional layer is equal to or lower than the respective amount of the first and/or the second active ingredients in the dosage form.

On the other hand it makes sense to choose a content of the first and/or the second active ingredients in the additional layer relative to the proportion of the first and/or the second active ingredients in the dosage form which is sufficiently high. It was shown that it is advantageous according to the present invention, when the mass ratio of the first active ingredient in the dosage form to the first active ingredient in the additional layer and/or the mass ratio of the second active ingredient in the dosage form to the second active ingredient in the additional layer do not exceed a value of 2.5:1, preferably a value of 2.2:1 and more preferably a value of 2.1:1. When the content of active ingredient in the additional layer is too low, then there is the risk that sufficient plasma levels of the first and the second active ingredients cannot be achieved, in particular in the case of immediate release of the first and the second active ingredients from the additional layer. Furthermore, with a sufficiently high content of the first and the second active ingredients in the additional layer the physiological retardation due to the residence time of the dosage form in the stomach can be advantageously used. Namely, due to the residence time in the stomach an additionally present additional layer is subject to a certain physiological retardation. When the content of the first and the second active ingredients in the additional layer is sufficiently high, then for example it may be possible to decrease the content of active ingredient in the dosage form according to the present invention in total. With that the size of the dosage form can be reduced in addition which may result in positive effects with respect to its swallowability and the compliance of the patient.

In an embodiment according to the present invention the mass ratio of the first active ingredient in the dosage form to the first active ingredient in the additional layer and/or the mass ratio of the second active ingredient in the dosage form to the second active ingredient in the additional layer are 2:1.

Furthermore, the additional layer may contain adjuvants, for example selected from fillers, pressing aids, binders, surfactants, fatty alcohols, triglycerides, antioxidants, disintegrants, complexing agents, coating agents, preservatives, softening agents, retarding agents, pigments and mixtures of such substances. A person skilled in the art is capable of choosing accordingly pharmaceutically acceptable adjuvants as well as suitable proportions of amounts.

When the additional layer should immediately release the first and the second active ingredients, then it was shown that proportions of adjuvants selected from fillers, disintegrants and pressing aids are suitable. The proportion of the adjuvants in the additional layer may be up to 95% by weight, more preferably up to 90% by weight and still more preferably up to 85% by weight. Proportions of adjuvants which are too high result in a total size of the additional layer which is too large.

Suitable fillers for an additional layer are such ones which at the same time show good compressibility. So the dosage form during pressing the additional layer on it must not be damaged. It was shown that fillers selected from microcrystalline cellulose, calcium hydrogenphosphate dihydrate and mixtures thereof are advantageous, wherein a proportion of between 40% by weight and 65% by weight of filler, based on the total mass of the additional layer, is favorable.

When the additional layer should immediately release the first and the second active ingredients, then it is advantageous, when a disintegrant is contained in the additional layer. Advantageous are proportions of between 10% by weight and 30% by weight. Suitable disintegrants are starches, starch derivatives or mixtures thereof. Pregelatinized starch such as Starch 1500, for example, is advantageous.

Pressing aids facilitate the production and processing of the additional layer and may in particular comprise lubricants such as highly disperse silica, magnesium stearate or mixtures thereof. Advantageous proportions of pressing aids of the additional layer are between 0.25% by weight and 5% by weight.

Thus, an additional layer may contain:
a. one or more active ingredients, in particular the first and the second active ingredients;
b. adjuvants, such as for example selected from fillers, disintegrants, pressing aids and mixtures thereof.

The invention relates also to a method for the production of the dosage form according to the present invention. A great advantage of the dosage forms according to the present invention is that they can be produced cheaply and quickly, thus the production method only requires few process steps with low expenditure.

The production method according to the present invention comprises the steps:
a. mixing of components of the dosage form;
b. granulating of the components; and
c. preparing a monolithic dosage form from the granulate.

At first components of the dosage form according to the present invention are mixed, preferably at least the first active ingredient, the second active ingredient, the emulsifier, the controlled-release agent and optionally the binder.

The mixing of the components preferably comprises the production of a so-called “mixture I” and a “mixture II”.

Mixture I comprises at least the first active ingredient and the emulsifier as well as optionally other components. Optionally, also a solvent may be contained. According to the exact constitution of the active ingredient and the emulsifier different solvents may be possible. Preferably, solvents which are common in pharmaceutical technology are used, in particular alcohols, esters and/or ketones, in particular acetone. Particularly preferably, mixture I consists of the first active ingredient and the emulsifier.

Mixture I can be produced by mixing of at least the first active ingredient and the emulsifier, wherein mixers which are known by a person skilled in the art such as for example a forced mixer can be used. Preferably, the mixing is conducted such that the emulsifier is molten and the first active ingredient and optionally further components are dissolved in the melt. In an alternative embodiment the first active ingredient and the emulsifier and optionally further components are mixed, in particular in a forced mixer, and subsequently they are molten.

Mixture II comprises the second active ingredient, the controlled-release agent and optionally further components, in particular the binder. Particularly preferably, mixture II consists of the second active ingredient, the optional binder and the controlled-release agent. Mixture II is produced by mixing of at least the second active ingredient, the controlled-release agent and optionally the binder. This can be conducted with mixers which are known by a person skilled in the art, such as for example a forced mixer.

Then the components of the dosage form according to the present invention are granulated for obtaining a granulate. This may be conducted such that mixture I and mixture II are granulated by granulation methods which are known by a person skilled in the art, in particularly selected from mixing granulation, melt granulation and spray granulation, such as for example as fluidized bed granulation.

In embodiments which are preferable according to the present invention a granulate is produced by means of a spray method, preferably by spraying mixture I onto mixture II, optionally using a solvent. Preferably, the spraying step is conducted in a fluidized bed apparatus. More preferably, the spraying step is conducted without the use of any solvent. It was shown that it is particularly advantageous to provide mixture II in a fluidized bed apparatus and subsequently to spray mixture I onto provided mixture II. Such a method allows a quick and cost-effective granulation step. The granulates obtained have optimally suitable sizes for further processing and very good flowing properties so that also the production of the monolithic dosage form from the granulate can be conducted in a quick and cost-effective manner.

In an alternative the granulation may be conducted such that mixture I is “granulated onto” mixture II. According to the present invention the “granulating onto”-step is conducted by ordinary mixing granulation in a well-established mixer or kneader-mixer. Also with such a granulation step a granulate which is suitable for the production of the dosage form according to the present invention can be obtained. The “granulating onto”-step is particularly advantageous according to the present invention, when granulates with particularly good coherence and good plasticity are desired.

In alternative embodiments the granulation of mixture I and mixture II is conducted by mixing of mixture I and mixture II, wherein a melt granulate is produced.

Then from the granulate a monolithic dosage form is produced. For that the granulate is preferably sieved, preferably with a screen-size opening of 1 mm for guaranteeing uniform granulate grains. This is also advantageous on the one hand for a uniform release and on the other hand for a good processability of the granulate.

The granulate which preferably has been sieved is processed with the optional lubricant and optional other adjuvants to a dosage form according to the present invention. In the case of a tablet the granulate is compressed in a tableting machine together with the optional lubricant and optional other adjuvants. Here, preferably a pressing force of at most 35 kN, more preferably at most 30 kN and particularly preferably at most 28 kN and especially preferably at most 18 kN is applied. When too high pressing forces are used, then often dosage forms with too high strength are obtained, which results in associated worse release of the active ingredients from the dosage form. But the pressing force should preferably exceed 7.5 kN, more preferably 8 kN. When too low pressing forces are used, then this results in insufficient strength of the dosage form.

Subsequently, the dosage form can optionally be provided with an additional layer, preferably by pressing an additional layer mixture comprising the components of the additional layer onto the dosage form. The additional layer mixture may also be present as a granulate which can be produced by granulating the components of the additional layer with granulation methods which are known by a person skilled in the art.

If required, the first and/or the second active ingredients can be comminuted prior to the production of the dosage form according to the present invention, in particular in a nano mill. The powder of the first active ingredient thus obtained is preferably incorporated into the emulsifier. For the incorporation a solvent is used in which the first active ingredient is not soluble, in particular water. The incorporation prevents the reagglomeration of the active ingredient particles.

### FIGURES

FIG. 1 shows the release of a first active ingredient (cinnarizine) and a second active ingredient (dimenhydrinate) from a dosage form according to the present invention according to example 1 in the fasting state. The measurement was conducted in 200 ml test medium by means of the Bio-Dis apparatus (25 dpm—dips per minute, 37° C.). The measurement of the amounts of active ingredient was conducted by means of HPLC. A clocked release profile was chosen for mimicking the fasting state in a manner which is as physiological as possible. Here as test media in the first 60 minutes 0.1 N HCl, followed by FaSSIF buffer according to Dressman et al. for 120 minutes, followed by 50% FaSSIF for 60 minutes and followed by buffer pH 7.0 for additional 60 minutes were chosen. The ordinate shows the released amount of active ingredient in percentages, the abscissa shows the elapsed minutes.

Within 60 minutes less than 20% of cinnarizine and diphenhydramine and/or 8-chlorotheophylline are released from the dosage form, based on the amount of active ingredient in the dosage form. After 120 minutes less than 30% are released from the dosage form. Also after 5 hours the release still persists. Thus, the dosage form releases the first and the second active ingredients in an optimally delayed and extended manner.

FIG. 2 shows the release of a first active ingredient (cinnarizine) and a second active ingredient (dimenhydrinate) from a dosage form according to the present invention according to example 1 in the saturated state. The measurement was conducted in 200 ml test medium by means of the Bio-Dis apparatus (25 dpm—dips per minute, 37° C.). The measurement of the amounts of active ingredient was conducted by means of HPLC. A clocked release profile was chosen for mimicking the saturated state in a manner which is as physiological as possible. Here as test media in the first 60 minutes FeSSGF according to Dressman et al., followed by buffer solution pH 3.0 R for 120 minutes, followed by 0.01 N HCl for 60 minutes and followed by FaSSIF according to Dressman et al. for additional 60 minutes were chosen. The ordinate shows the released amount of active ingredient in percentages, the abscissa shows the elapsed minutes.

Within 60 minutes less than 20% of cinnarizine and diphenhydramine and/or 8-chlorotheophylline are released from the dosage form. After 120 minutes less than 30% are released from the dosage form. Also after 5 hours the release still persists. Thus, the dosage form releases the first and the second active ingredients in an optimally delayed and extended manner, even in the saturated state.

### EXAMPLES

### Example 1

A dosage form with the following composition was prepared:

| | Amount | |
|---|---|---|
| Constituent | (mg) | Function |
| cinnarizine | 60 | first active ingredient |
| dimenhydrinate | 120 | second active ingredient |
| Methocel ® E4M | 240 | controlled-release agent |
| (hydroxypropylmethylcellulose) | | |
| Gelucire ® 50/13 | 240 | emulsifier |
| lactose, anhydrous | 120 | binder |
| total mass: | 780 | |

A dosage form according to the present invention with the active ingredient cinnarizine (first active ingredient) and the further active ingredient dimenhydrinate (second active ingredient) was prepared. Cinnarizine is a weak base which at a pH value of 1 is characterized by a water solubility of about 1.55 mg/ml. The solubility at a pH value of above 7 is lower than 0.01 mg/ml. At a pH value of 7, for example, the solubility is lower than about 0.00025 mg/ml. Thus, according to the present invention cinnarizine is an active ingredient having low solubility which in addition is protonated in the acidic pH range of the stomach and virtually insoluble in the pH range of the intestine. Furthermore, cinnarizine comprises two amino groups. Thus, cinnarizine is a first active ingredient according to the present invention. In addition, cinnarizine shows only limited storage stability and its storage requires protection from light, which is an additional challenge for the formulation of cinnarizine.

At a pH value of 7 dimenhydrinate is characterized by a higher solubility than cinnarizine. At a pH value of 7 the solubility is at least one decimal power higher than the solubility of cinnarizine. Thus, dimenhydrinate is a second active ingredient according to the present invention. Dimenhydrinate has also permanent charges. In addition, dimenhydrinate has a bitter and anesthetic taste, which is an additional challenge for the formulation of dimenhydrinate.

For the production of the dosage form the mentioned components were processed in accordance with the production method according to the present invention. At first, the components were mixed, wherein a mixture I and a mixture II were prepared. Mixture I was prepared by melting of the emulsifier (Gelucire® 50/13) and dissolving cinnarizine as the first active ingredient in the melt. Mixture II was obtained by mixing dimenhydrinate as the second active ingredient with the controlled-release agent (Methocel® E4M) and the binder (lactose, anhydrous). Subsequently, the components were granulated by granulating mixture I onto mixture II. Subsequently, the granulate obtained was compressed into a tablet.

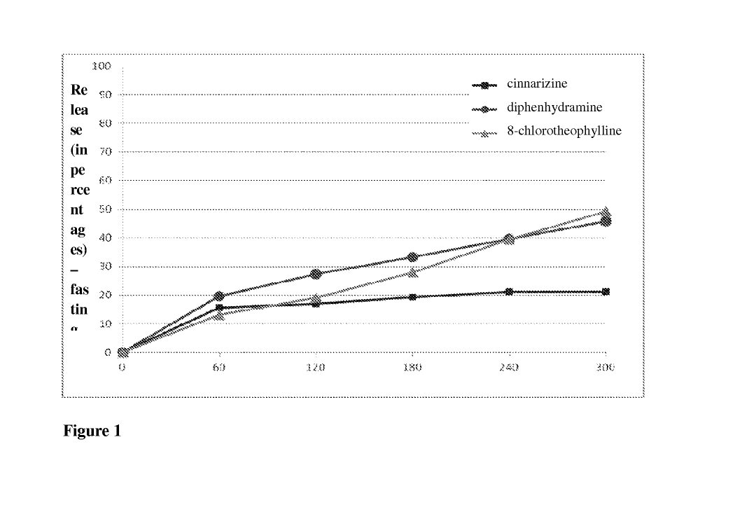

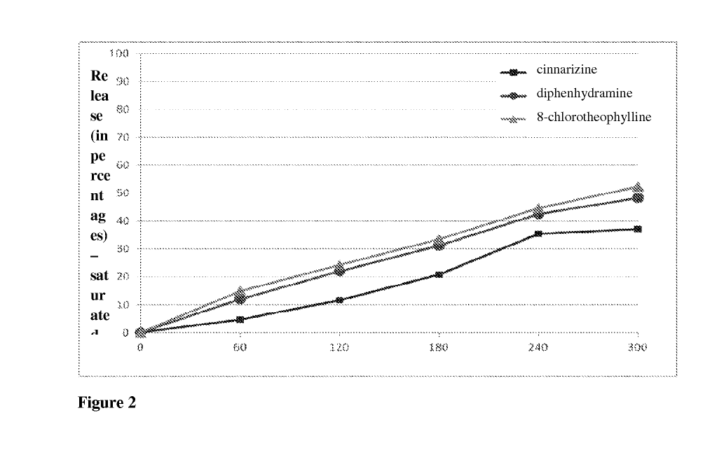

The FIGS. 1 and 2 show the release of the first and the second active ingredients from the dosage form, wherein a clocked release profile was chosen for mimicking the physiological states and processes in a manner which is as exactly as possible. Here, a fasting state (FIG. 1) and a saturated state (FIG. 2) were mimicked. It can be seen that the release slowly increases in both cases, in the fasting and also in the saturated state, so that an extended effect can be achieved. The risk of dose dumping does also not exist in the saturated state. Even in the case of a residence time of the dosage form in the stomach in the saturated state of four hours less than 36% of cinnarizine, less than 45% of diphenhydramine and less than 45% of 8-chlorotheophylline are released from the dosage form according to the present invention. Therefore, such an increase of the plasma levels in both cases, the intake of the dosage form in the fasting and also in the saturated state, can be expected that the daily intake can be reduced to two dosage forms, preferably 1 dosage form per day, which is conducive to an improvement of the compliance of the patient.

### Example 2

A dosage form with the following composition was prepared:

| | Amount | |
|---|---|---|
| Constituent | (mg) | Function |
| cinnarizine | 60 | first active ingredient |
| dimenhydrinate | 120 | second active ingredient |
| Methocel ® E4M | 240 | controlled-release agent |
| (hydroxypropylmethylcellulose) | | |
| Gelucire ® 50/13 | 240 | emulsifier |
| lactose, anhydrous | 120 | binder |
| total mass: | 780 | |

For the production of the dosage form the mentioned components were processed in accordance with the production method according to the present invention. At first, the components were mixed, wherein a mixture I and a mixture II were prepared. Mixture I was prepared by melting of the emulsifier (Gelucire® 50/13) and dissolving cinnarizine as the first active ingredient in the melt. Mixture II was obtained by mixing dimenhydrinate as the second active ingredient with the controlled-release agent (Methocel® E4M) and the binder (lactose, anhydrous).

Subsequently, the components were granulated by spraying mixture I onto provided mixture II in a fluidized bed apparatus. The granulate obtained showed optimal properties for further processing, in particular with respect to the flowing properties and the size of the granulate grains, and subsequently it was possible to easily and quickly compress it into a tablet.

### Example 3

A dosage form with the following composition was prepared:

| | Amount | |
|---|---|---|
| Constituent | (mg) | Function |
| cinnarizine | 60 | first active ingredient |
| dimenhydrinate | 120 | second active ingredient |
| Methocel ® E4M | 240 | controlled-release agent |
| (hydroxypropylmethylcellulose) | | |
| Gelucire ® 50/13 | 240 | emulsifier |
| corn starch | 120 | binder |
| Aerosil ® (highly disperse silica) | 7.8 | lubricant |
| magnesium stearate | 15.6 | lubricant |
| talcum | 31.2 | lubricant |
| total mass: | 834.6 | |

Here, the mentioned components were processed in accordance with the production method according to the present invention. At first, the components were mixed, wherein a mixture I and a mixture II were prepared.

Mixture I was prepared by melting of the emulsifier (Gelucire® 50/13) and dissolving cinnarizine as the first active ingredient in the melt. Mixture II was obtained by mixing dimenhydrinate as the second active ingredient with the controlled-release agent (Methocel® E4M) and the binder (corn starch). Subsequently, the components were granulated by granulating mixture I onto mixture II. Subsequently, the granulate obtained together with the lubricant mixture were compressed into a tablet.

### Example 4

A dosage form with the following composition was prepared:

| | Amount | |
|---|---|---|
| Constituent | (mg) | Function |
| cinnarizine | 40 | first active ingredient |
| dimenhydrinate | 80 | second active ingredient |
| Methocel ® E4M | 160 | controlled-release agent |
| (hydroxypropylmethylcellulose) | | |
| Gelucire ® 50/13 | 160 | emulsifier |
| Pharmatose ® (anhydrous lactose) | 80 | binder |
| Aerosil ® (highly disperse silica) | 5.2 | lubricant |
| magnesium stearate | 10.4 | lubricant |
| talcum | 20.8 | lubricant |
| total mass: | 556.4 | |

Here, the mentioned components were processed in accordance with the production method according to the present invention. At first, the components were mixed, wherein a mixture I and a mixture II were prepared.

Mixture I was prepared by melting of the emulsifier (Gelucire® 50/13) and dissolving cinnarizine as the first active ingredient in the melt. Mixture II was obtained by mixing dimenhydrinate as the second active ingredient with the controlled-release agent (Methocel® E4M) and the binder (Pharmatose®). Subsequently, the components were granulated by granulating mixture I onto mixture II. Subsequently, the granulate obtained together with the lubricant mixture were compressed into a tablet.

For preparing the dosage form according to the present invention during compression the dosage form was provided with an additional layer having the following composition:

| | Amount | |
|---|---|---|
| Constituent | (mg) | Function |
| cinnarizine | 20 | first active ingredient |
| dimenhydrinate | 40 | second active ingredient |
| Starch 1500 (pregelatinized starch) | 50 | disintegrant |
| Vivapur ® 101 (microcrystalline | 80 | filler |
| cellulose) | | |
| Aerosil ® 200 (highly disperse silica) | 1.25 | pressing aid |
| magnesium stearate | 2.0 | pressing aid |
| Emcompress ® (calcium | 56.75 | filler |
| hydrogenphosphate dihydrate) | | |
| total mass: | 250 | |

For the production of the additional layer the components were mixed together and pressed onto the dosage form.

1. A monolithic dosage form containing
at least one first active ingredient and one second active ingredient, wherein the first active ingredient at a pH value of 7 and 22° C. has a solubility of lower than 0.01 mg/ml and wherein the second active ingredient at a pH value of 7 and 22° C. has a solubility which exceeds the solubility of the first active ingredient at a pH value of 7 and 22° C. by at least one decimal power, and
at least one emulsifier, wherein the emulsifier has a structural motif of the following formula
R¹—O—[CH₂—CH₂—O]ₙ—R²
wherein R¹ and R² independently from each other are hydrogen, alkyl, glyceride or polyalkylene oxide and n is an integer of at least 4, and
at least one controlled-release agent,
wherein the sum of the proportions of the controlled-release agent and the emulsifier of the dosage form is at least 40% by weight and wherein the mass ratio of the second active ingredient to the controlled-release agent is lower than 1:1.

2. The dosage form according to claim 1, wherein the sum of the proportions of the controlled-release agent and the emulsifier of the dosage form is at least 48% by weight.

3. The dosage form according to claim 1, wherein the first active ingredient is present in a mass in relation to the mass of the emulsifier of at most 1:2.

4. The dosage form according to claim 1, wherein the controlled-release agent is selected from methylcellulose, hydroxypropylmethylcellulose and mixtures thereof.

5. The dosage form according to claim 1, wherein the first active ingredient comprises at least one amino group and at a pH value of 1 and 22° C. has a solubility of at least 0.5 mg/ml.

6. The dosage form according to claim 1, wherein the second active ingredient has a half-life of elimination of longer than 2 hours.

7. The dosage form according to claim 1, wherein the dosage form is designed as a form with long residence time in the stomach which resides in the stomach for at least 2 hours, before it enters the intestine.

8. The dosage form according to claim 1, wherein the dosage form is designed as a tablet and wherein the tablet has a hardness of at least 40 N and at most 130 N.

9. A method for the production of a dosage form according to claim 1 with the steps:
a. mixing of components of the dosage form;
b. granulating of the components; and
c. preparing of a monolithic dosage form from the granulate.

10. (canceled)

11. A method of treating a subject, comprising administering to the subject the dosage form of claim 1.
